(19) United States
(12) Patent Application Publication
(10) Pub. No.: US 2016/0228639 A1
(43) Pub. Date: Aug. 11, 2016

(54) SYSTEM AND METHOD OF GIVING AN INJECTION OF RINGER

(71) Applicant: Hyeon Cheol ZIN, Gyeongsangbuk-do (KR)

(72) Inventor: Hyeon Cheol ZIN, Gyeongsangbuk-do (KR)

(21) Appl. No.: 14/862,058

(22) Filed: Sep. 22, 2015

(30) Foreign Application Priority Data
Feb. 10, 2015 (KR) 10-2015-0020027

(51) Int. Cl. A61M 5/172; A61M 5/168

(52) U.S. Cl. CPC A61M 5/1723; A61M 5/16895; A61M 5/16813; A61M 5/16877; A61M 2230/04; A61M 2230/50; A61M 2230/30; A61M 2205/3592; A61M 2205/18; A61M 2205/50

(57) ABSTRACT

The present invention relates to a system and method of giving an injection of Ringer which can grasp the living body data of the patient and the Ringer remainder data and automatically control the injection amount or the injection speed of the Ringer, or can inform this to the medical team or the protector through an external instrument and immediately change the injection condition of the Ringer according to the condition of the patient.

The system and method of giving an injection of Ringer according to the present invention can comprise: a weight estimation section for measuring the weight of the Ringer bag or estimating the remainder of the Ringer; a control section for receiving the data from the weight estimation section and judging and setting an injection amount or an injection speed of the Ringer; and a Ringer tube opening and closing adjustment section installed at a Ringer tube connected to the Ringer bag and adjusting the injection amount or the injection speed of the Ringer by a signal received from the control section. In addition, the control section can comprise a first communication section for controlling an external instrument or an opening and closing adjustment section or for transmitting or receiving a control signal to or from the external instrument or the opening and closing adjustment section, and the opening and closing adjustment section can comprise a second communication section for transmitting or receiving the control signal to or from the external instrument or the control section.

### BACKGROUND

(a) Technical Field

The present invention relates to a system and method of giving an injection of Ringer, and more particularly, it relates to a system and method of giving an injection of

Ringer which can grasp the living body data of the patient and the Ringer remainder data and automatically control the injection amount or the injection speed of the Ringer, or can inform this to the outside and immediately change the injection condition of the Ringer.

(b) Background Art

In general, the medicine is injected by using the Ringer bag or the Ringer container in case where a lot of medicine is necessary for the patient in the hospital. Since the Ringer bag recently used is made of vinyl material (Non PVC), there is a disadvantage in that as the Ringer is injected, the void space of the Ringer bag becomes depressurized condition, so that the envelopes of the Ringer bag attach to each other whereby the Ringer injection amount and the remainder amount are not precisely seen.

Furthermore, the scale indication spacing differs for each Ringer bag manufacturing company, and the scale does not consist with the Ringer amount so as to present difficulty to the nurse working at the site.

The Ringer injection procedure requires long time and in case where the amount of the Ringer injected per unit hour is large, the shock can be generated, and in addition, in case where after all the Ringer is injected, the air flows into the blood vessel, there can be risk detrimental to the patient. Therefore, there is a problem in that the medical team or the protector shall always observe the injection situation of the Ringer and the condition of the patient during the Ringer is injected.

Recently, there is the serious nurse lacking phenomenon in the hospital and in such nurse lacking condition, the works such as measuring the body temperature and blood pressure which are not difficult and the works such as replacement and measurement of the Ringer consume the time and the personal are not appropriately used.

To remove such inconveniences, in the conventional technologies, many patent applications are presented in which the Ringer injection amount is monitored and adjusted, and when the Ringer injection is completed the warning signal is outputted.

However, in such conventional technologies, the Ringer injection to suit the condition of the patient is not carried out based on the living body data of the patient. In addition, in case where the medical team or the protector is not present during the Ringer injection, the Ringer injection condition cannot be immediately changed. Furthermore, there is a problem in that the Ringer remainder limit set value cannot be set, and such set value cannot be warned by step and cannot be informed to the medical team or the protector.

Therefore, the present applicant devised the present invention to solve such problems, and there is the Korea registered patent publication 10-1413945 (Title of the invention: the Ringer amount sensing device, published date: 2014/06/30) as the prior art document related thereto.

### SUMMARY OF THE DISCLOSURE

The present invention has been made in an effort to solve the above-described problems, and can provide a system and method of giving an injection of Ringer which can grasp the Ringer remainder data and the living body data of the patient and estimate the appropriate Ringer injection amount or the injection speed based thereon in real time, and at the same time automatically control the injection amount or the injection speed of the Ringer remotely through the external instrument by using the radio communication method such as the Bluetooth, WIFI or the like.

The system and method of giving an injection of Ringer according to the present invention to accomplish the object described above can comprise: a weight estimation section for measuring the weight of the Ringer bag or estimating the remainder of the Ringer; a control section for receiving the data from the weight estimation section and judging and setting an injection amount or an injection speed of the Ringer; and a Ringer tube opening and closing adjustment section installed at a Ringer tube connected to the Ringer bag and adjusting the injection amount or the injection speed of the Ringer by a signal received from the control section.

The control section can comprise a first communication section for controlling an external instrument or an opening and closing adjustment section or for transmitting or receiving a control signal to or from the external instrument or the opening and closing adjustment section, and the opening and closing adjustment section can comprise a second communication section for transmitting or receiving the control signal to or from the external instrument or the control section.

The system can further comprise a data collection section for collecting living body data of the patient by being connected via wire or wirelessly to the body of the patient and for transmitting the collected living body data to the control section, and the data collection section can collect the living body data including the pulse, temperature, electrocardiogram or blood pressure of the patient and transmit it to the control section.

The control section can estimate in real time the injection amount or the injection speed of the Ringer appropriate to the patient in consideration of a Ringer remainder data measured by the weight estimation section or the living body data measured by the data collection section.

The opening and closing adjustment section can comprise a motor actuated according to the Ringer remainder data or the living body data, and a tube opening and closing section which is installed at the Ringer tube so as to adjust the injection amount or the injection speed of the Ringer.

The external instrument can be provided as a radio communication instrument and receive the input data set by the medical team or the protector, and the first communication section or the second communication section can receive the input data and manually adjust the injection amount or injection speed of the Ringer.

The control section can set the primary limit or the secondary limit about the Ringer remainder or the remainder weight and send the warning signal to the external instrument by stages in case where the Ringer is equal to or below the primary limit or the secondary limit.

The control section can comprise the priority setting section which receives the input data from the medical team or the protector during the Ringer injection and discriminates the priority among the input data and the Ringer remainder data measured by the weight estimation section.

The priority setting section, in case where the input data are input to the external instrument during the preset time after the warning signal is generated, can preferentially process the input data, and in case where the input data are not input to the external instrument during the preset time, can automatically close the tube opening and closing section or reduce the injection speed of the Ringer by the control section.

The living body data of the patient, the remainder amount of the Ringer, the injection speed, the injection completion expectation time or the medical team in charge can be displayed on the external instrument 150, and at least one among the remainder amount of the Ringer, the injection speed of the Ringer, the injection completion expectation time of the Ringer can be adjusted by the setting by the protector or the medical team.

The system can further comprise a data storage section for storing the living body data or the Ringer remainder data transmitted by the first communication section or the second communication section.

The opening and closing adjustment section can further comprise an auxiliary medicine removal/attachment section providing the medicine or injection necessary for the patient, and the auxiliary medicine removal/attachment section can be controlled through the external instrument or by the medical team, protector, or patient.

A method of giving an injection of Ringer using the system of giving an injection of Ringer according to the present invention can comprise: a weight estimation step for measuring the weight of the Ringer bag or estimating the Ringer remainder by a weight estimation section; a data collection step for collecting the living body data of the patient by being connected to the body of the patient by wire or wirelessly; a control step for setting the injection amount or the injection speed of the Ringer by using the data received from the weight estimation step and the data collection step; an actuation step for adjusting the actuation condition of an opening and closing adjustment section installed at a Ringer tube according to the injection amount or the injection speed of the Ringer estimated by the control step; and a setting step in which the injection amount or the injection speed of the Ringer is set by receiving the input data from an external instrument.

The control step can control the injection amount or the injection speed of the Ringer to suit the patient based on the data obtained by the weight estimation step or the data collection step.

The control step can set a primary limit and a second limit according to the Ringer remainder or the remainder weight, and the control step can comprise a warning step for warning the external instrument in case where the Ringer remainder or the remainder weight is equal to or below the primary limit or the secondary limit, and a priority discriminating step for discriminating the priority among the input data set by the setting step and the Ringer remainder data estimated by the weight estimation step.

The priority discriminating step, in case where the input data are input to the external instrument during the preset time after the warning step, can preferentially process the input data, and in case where the input data are not input to the external instrument during the preset time, can automatically close the tube opening and closing section or reduce the injection speed of the Ringer by the control step.

### Effect of the Invention

The present invention as described above can inject the Ringer efficiently to suit the patient by setting or changing the Ringer injection amount or the injection speed based on the weight data of the Ringer injected to the patient or the living body data obtained by measuring the body condition of the patient.

In addition, the present invention can prevent the medical accident by rapidly adjusting or setting the injection condition including the injection amount or the injection speed of the Ringer remotely from the external instrument.

In addition, the present invention can enable the protector or the medical team to perform the service more efficiently than the conventional one and can provide the patient with the safe medical service by setting the warning signal by steps by inputting the limit of the Ringer in advance.

### BRIEF DESCRIPTION OF THE DRAWINGS

FIG. 1 is a drawing showing the basic structure of a system of giving an injection of Ringer according to an embodiment of the present invention;

FIG. 2 is a block diagram of a system of giving an injection of Ringer according to an embodiment of the present invention;

FIG. 3 is a partial perspective view concretely showing a weight estimation section, a control section, and a drive section by enlarging A portion of FIG. 1;

FIG. 4 is a drawing showing in detail a screen of an external instrument by enlarging B portion of FIG. 1; and

FIG. 5 is a flow diagram showing a method of giving an injection of Ringer according to an embodiment of the present invention.

### DETAILED DESCRIPTION

The advantages and/or the characteristics of the present invention and the method for accomplishing them will be obvious by referring to the embodiments described below in detail together with the accompanying drawings. However, the present invention is not limited to the embodiments disclosed below but will be embodied in various forms differing from each other, and the present embodiments are provided to make the disclosure of the present invention to be complete and to completely inform those skilled in the art of the scope of the invention, and the present invention is defined only by the scope of the claims of the present invention. The identical reference numbers refers to identical construction elements through the entire specification.

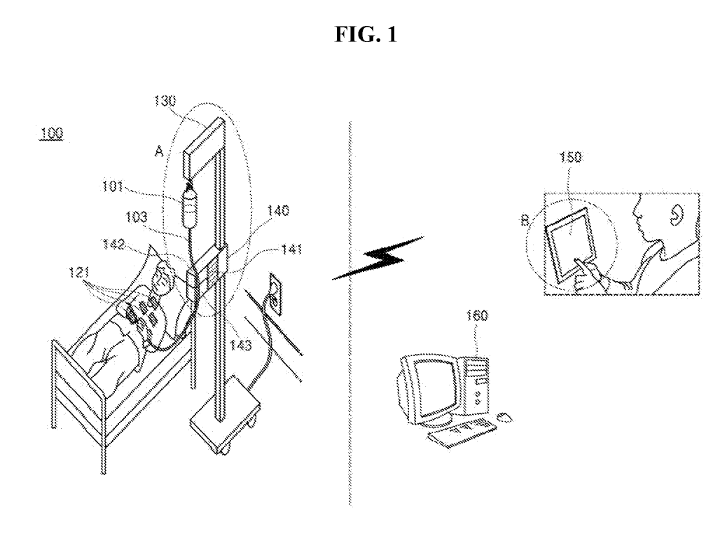

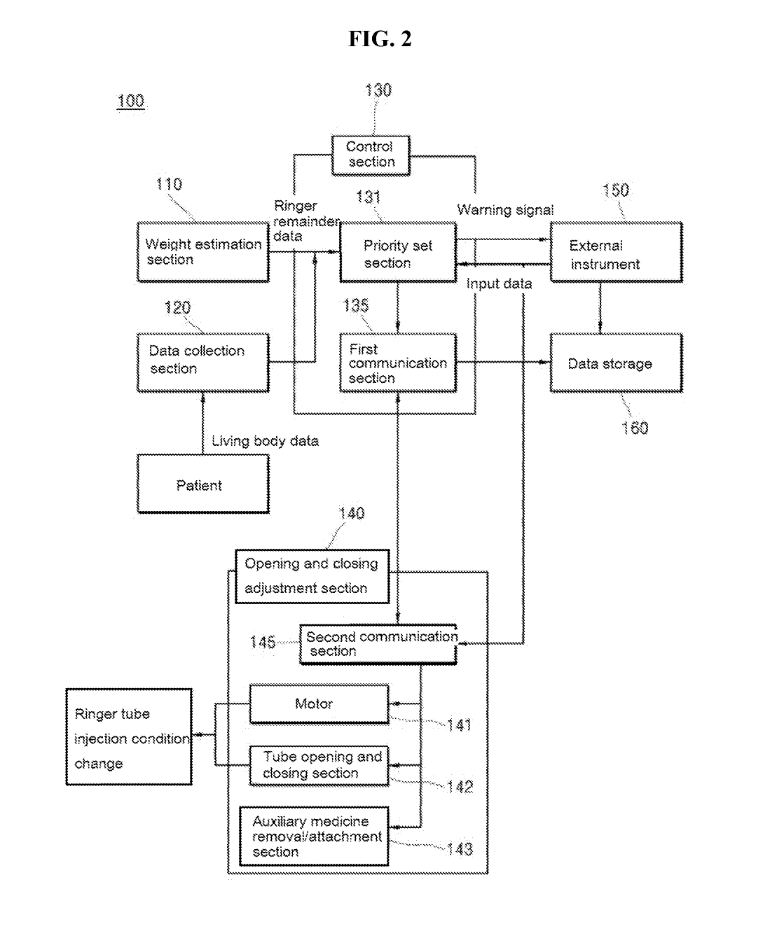

FIG. 1 is a drawing showing the basic structure of a system 100 of giving an injection of Ringer according to an embodiment of the present invention, and FIG. 2 is a block diagram of a system 100 of giving an injection of Ringer according to an embodiment of the present invention.

The system 100 of giving an injection of Ringer according to an embodiment of the present invention can be used after hang a Ringer bag 101 and a Ringer tube 103 on a conventional Ringer hanging stand generally provided at a hospital and the like. In addition, a conventional Ringer hanging stand is used only as a hanging stand necessary for supplying a simple Ringer, however, the system 100 of giving an injection of Ringer according to an embodiment of the present invention can be used as a patient monitoring and transmitting device which can monitor the condition of the patient and transmit to a server.

As shown in FIGS. 1 and 2, the system 100 of giving an injection of Ringer according to the present invention can comprise: a weight estimation section 110 for measuring the weight of the Ringer bag 101 or estimating the remainder of the Ringer; a control section 130 for receiving the data from the weight estimation section 110 and judging and setting an injection amount or an injection speed of the Ringer; and a Ringer tube opening and closing adjustment section 140 installed at a Ringer tube 103 connected to the Ringer bag 101 and adjusting the injection amount or the injection speed of the Ringer by a signal received from the control section 130.

At this time, the control section 130 can comprise a first communication section 135 for controlling an external instrument 150 or an opening and closing adjustment section 140 or for transmitting or receiving a control signal to or from the external instrument 150 or the opening and closing adjustment section 140, and the opening and closing adjustment section 140 can comprise a second communication section 145 for transmitting or receiving the control signal to or from the external instrument 150 or the control section 130.

The present invention is provided with the first communication section 135 of the control section 130 and the second communication section 145 of the opening and closing adjustment section 140 so as to be able to transmit or receive the signal between the first communication section 135, the second communication section 145, and the external instrument 150.

The system 100 of giving an injection of Ringer according to the present invention can further comprise a data collection section 120 for collecting living body data of the patient by being connected via wire or wirelessly to the body of the patient and for transmitting the collected living body data to the control section 130. At this time, the data collection section 120 can collect the living body data including the pulse, temperature, electrocardiogram or blood pressure of the patient and transmit it to the control section 130.

The data collection section 120 can be attached to the body of the patient so as to transmit the various living body data to the control section 130. At this time, the living body data can further include more data if required by a medical team to cure the patient besides the pulse, temperature, electrocardiogram or blood pressure of the patient.

The control section 130 can estimate in real time the injection amount or the injection speed of the Ringer appropriate to the patient in consideration of a Ringer remainder data measured by the weight estimation section 110 or the living body data measured by the data collection section 120.

A reference number “121” not noted in FIG. 1 is pads for collecting the living body data.

The Ringer remainder data can be received from the weight estimation section 110 and the living body data can be received from the data collection section 120, and the two kinds of data can be transmitted to the control section 130. The control section 130 can estimate the injection amount or the injection speed of the Ringer appropriate to the patient based on the received data and transmit it the opening and closing adjustment section 140 so as to adjust the degree of opening and closing of the Ringer tube 103.

Describing the flow of movement of data, the Ringer remainder data and the living body data received by the control section 130 are transmitted by the first communication section 135, and the opening and closing adjustment section 140 can receive the data estimated based on the Ringer remainder data and the living body data from the second communication section 145 and adjust the degree of opening and closing of the Ringer tube 103 in real time so as to be able to adjust the injection amount or the injection speed of the Ringer.

Therefore, the system 100 of giving an injection of Ringer according to the present invention can check the Ringer remainder data and the living body data in real time and control, based on this, the opening and closing adjustment section 140 so as to smoothly change the injection amount or the injection speed of the Ringer.

In addition, the system 100 of giving an injection of Ringer according to the present invention can further comprise a power supply (not shown) for supplying the electric power necessary for each constructional element.

In addition, the system 100 of giving an injection of Ringer according to the present invention can further comprise a display (not shown) for displaying the information on the Ringer or the patient such as the Ringer remainder data, Ringer injection completion expectation time, Ringer injection speed, living body data or the like.

By providing a structure of the display in addition to the external instrument 150, efficient work performance and safety medical service can be provided by enabling the patient, medical team or protector to directly confirm the information on the Ringer or patient visually through the structure of the display.

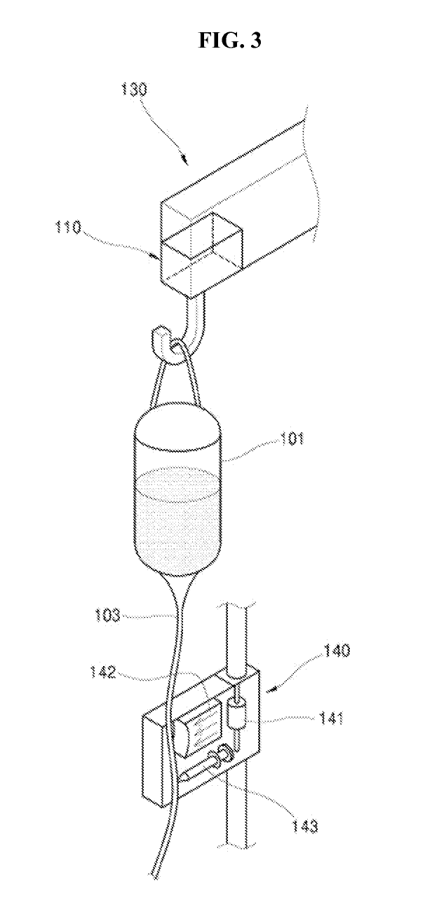

FIG. 3 is a partial perspective view concretely showing the weight estimation section 110, the control section 130, and the opening and closing adjustment section 140 by enlarging a portion of FIG. 1. As shown in FIG.3, the weight estimation section 110 can measure the weight of the Ringer bag 101 or estimate the remainder of the Ringer. The weight estimation section 110 can measure the weight of the Ringer bag 101 and estimate the data of the remainder of the Ringer in real time, and signalize and transmit them to the control section 130.

The system 100 of giving an injection of Ringer according to the present invention can use a weight measurement sensor (not shown) to measure the weight of the Ringer remaining in the Ringer bag 101. The weight measurement sensor can be provided in the weight estimation section 110. The weight measurement sensor can be formed in a form of hanging the Ringer bag 101, or can obtain the weight of the Ringer bag 101 or the Ringer remainder data by being connected to the structure from which the Ringer bag 101 is hung.

As the weight measurement sensor, a spring sensor, infrared ray sensor, optical sensor, magnetic field sensor, liquid sensor, camera or load cell (weight sensor) can be used to measure the weight. Therefore, although the present invention uses the load cell to measure the weight of the Ringer bag 101 or the Ringer remainder data, however, is not limited thereto and other sensors can be used in the weight estimation section 110.

The opening and closing adjustment section 140 can comprise a motor 141 actuated according to the Ringer remainder data or the living body data, and a tube opening and closing section 142 which is actuated by the motor 141 and which adjusts the injection amount or the injection speed of the Ringer by pressurizing the Ringer tube 103.

The opening and closing adjustment section 140 can further comprise an auxiliary medicine removal/attachment section 143 providing the medicine or injection necessary for the patient, and the auxiliary medicine removal/attachment section 143 can be controlled through the external instrument 150 or by the medical team, protector, or patient.

The opening and closing adjustment section 140 can comprise the motor 141 and the tube opening and closing section 142 to adjust the degree of opening and closing of the fluid passage through which the Ringer of the Ringer tube 103 flows. At this time, in addition to the motor 141 and the tube opening and closing section 142, the auxiliary medicine removal/attachment section 143 can be installed proximally at one side so as to provide the medicine as necessary according to the condition of the patient and can control this.

At this time, the tube opening and closing section 142 is actuated by the motor 141, so as to control the flow of the Ringer flowing in the Ringer tube 103 thereby control the injection amount or injection speed of the Ringer.

In addition, the tube opening and closing section 142 can be formed by a rack-pinion, lead screw, linear compression bar, valve or plate or the like, is not limited to this, and any means which can adjust the flow of the Ringer flowing in the Ringer tube 103 can be used.

In the auxiliary medicine removal/attachment section 143, a syringe which includes an auxiliary medicine which should be injected without fail to the patient in addition to the Ringer can be stored. In case the medical team should inject the auxiliary medicine to the patient, they can set the auxiliary medicine injection time or injection amount through the external instrument 150.

The external instrument 150 can be provided as a radio communication instrument so as to receive the input data set by the protector of the patient or the medical team, or the first communication section 135 or the second communication section 145 can receive the input data and manually adjust the injection amount or injection speed of the Ringer.

Therefore, in the present invention, the control section 130 estimates the injection amount or injection speed of the Ringer appropriate to the patient provided by the weight estimation section 110 and the data collection section 120 and automatically adjusts this, or in case where the input data are input by the external instrument 150, the injection amount or injection speed of the Ringer can be manually adjusted by the medical team or the protector.

The first communication section 135, in case where the Ringer remainder data is equal to or below the primary limit or the secondary limit of the Ringer remainder, can send a warning signal to the external instrument 150 or can receive the input data input by the external instrument 150. At this time, the data of the injection amount or injection speed of the Ringer changed by the control section 130 can be sent to the second communication section 145.

That is, the second communication section 145 can receive the data of the injection amount or injection speed of the Ringer estimated by the control section 130.

The external instrument 150 receives the Ringer remainder data or the warning signal transmitted from the first communication section 135, and thereafter the medical team or the protector can input the input data and again send the instruction to the first communication section 135.

Therefore, the system 100 of giving an injection of Ringer according to the present invention transmits in real time the weight data periodically through the communication network upon the decrease of the weight according to the injection of the Ringer, whereby the server can immediately monitor the injection situation of the Ringer. In addition, there is an advantage in that in case where an abnormal injection occurs, the warning signal is generated so that the medical team can efficiently manage the patient.

The system 100 of giving an injection of Ringer according to the present invention can further comprise a data storage section 160 for storing the living body data or the Ringer remainder data transmitted by the first communication section 135 or the second communication section 145.

Specifically, the data storage section 160 can store the Ringer related data including the history of injection of the Ringer or the medicine to the patient or the like, the living body data obtained from the patient, or the input data input from the external instrument 150. Accordingly, the entire confirmation as the contents of personal history of the patient or the injection data becomes possible.

The data transmitted or received between the first communication section 135, the second communication section 145, and the external instrument 150 can be transferred to the data storage section 160. The first communication section 135, the second communication section 145, and the external instrument 150 can use a ZigBee, radio LAN module, Bluetooth, WIFI, UWB or the like as a radio communication module for collecting the data.

The operation of the system 100 of giving an injection of Ringer according to the present invention will be concretely described below.

The system 100 of giving an injection of Ringer according to the present invention can set the limit of the Ringer, compare the Ringer remainder and the limit, send the warning signal of the result to the outside by stages. Thereafter, the medical team or the protector confirms this, and then compare the input data and the Ringer remainder data estimated by the weight estimation section 110, and provide the injection amount or injection speed most preferable for the patient based on the preset information.

Therefore, the present invention estimates in real time the data of the injection and sends the warning to the external instrument 150, so that the medical team or the protector can rapidly adjust or set the injection condition at the external instrument 150 remotely. A priority setting section 131 discriminates the priority among the set input data and the Ringer remainder data, and actuates the opening and closing adjustment section 140 through the discriminated data so as to adjust the injection amount or injection speed of the Ringer to be appropriate to the patient.

The control section 130 can set the primary limit or the secondary limit about the Ringer remainder or the remainder weight, and can send the warning signal to the external instrument 150 by stages in case where the Ringer is equal to or below the primary limit or the secondary limit.

At this time, the medical team or the protector having received the warning signal can input the input data through the external instrument 150. Accordingly, the control section 130 can receive the input data transmitted from the external instrument 150.

That is, the control section 130 can generate the first step or second step warning signal about the primary limit or secondary limit and transmit it to the external instrument 150 in case where the remainder of the Ringer or the remainder weight is equal to or below the primary limit or the secondary limit.

The primary limit or secondary limit about the remainder of the Ringer or the remainder weight can be flexibly set by the medical team for each patient without limitation of the place or the time according to the experience or the work situation of the medical team or the condition of the patient. In addition, the primary limit or secondary limit about the remainder of the Ringer or the remainder weight can be set as %, cc, weight or time based on the remained Ringer remainder data.

The warning signal plays a role of informing the protector or the medical team of the lack of the Ringer remainder, and carries out the warning sound, warning comment or warning indication or provides visually the information on the Ringer remainder.

In addition, the control section 130 can immediately set the warning signal to the medical team or the protector in advance in case where the patient is in emergency situation (power failure, instability of the patient's pulse, or the like) or a problem about the Ringer injection occurs, besides the case where the remainder of the Ringer or the remainder weight is equal to or below the primary limit or the secondary limit.

The control section 130 can comprise the priority setting section 131 which receives the input data input to the external instrument 150 by the medical team or the protector and discriminates the priority among the input data or the Ringer remainder data.

That is, the control section 130 can comprise the priority setting section 131 which discriminates the priority among the input data input to the external instrument 150 by the medical team or the protector or the Ringer remainder data measured by the weight estimation section 110.

In the system 100 of giving an injection of Ringer according to the present invention, the control section 130, during the injection of the Ringer, can receive in real time the input data input to the external instrument 150 by the medical team or the protector (manual manner), or receive in real time the Ringer remainder data provided by the weight estimation section 110 (automatic manner). The priority setting section 131 of the control section 130 can discriminate the priority among these data based on the preset information and determine the injection amount or injection speed of the Ringer appropriate to the patient.

In the meantime, at the time of generation of the warning signal, the cases may be classified into a case where the medical team or the protector response within the preset time after the warning signal and a case where the medical team or the protector does not response. That is, the operation can be classified into a case where the input data are input and a case where the input data are not input.

After the warning signal is generated, the priority setting section 131 can preferentially process the input data directly input by the medical team or the protector in case where the input data are input to the external instrument 150 during the preset time. On the other hand, the priority setting section 131 can automatically close the tube opening and closing section 142 or reduce the injection speed of the Ringer by the control section 130 in case where the input data are not input from the external instrument 150 during the preset time.

That is, if the medical team or the protector cannot input the input data through the external instrument 150 within the preset time after the warning signal is sent to the external instrument 150, the control section 130 can automatically recognize this as an emergency situation and close the tube opening and closing section 142 or reduce the injection speed of the Ringer based on the Ringer remainder data. This can prevent the danger of the air going into the blood vessel after all the Ringer is injected by immediately interrupting by force the Ringer injected to the patient or by reducing the injection speed.

Therefore, the system 100 of giving an injection of Ringer according to the present invention can interrupt the injection while all of Ringer is not injected but a certain amount is remained by the warning signal for the primary or the secondary step about the preset primary limit or secondary limit. That is, there is an advantage in that since it is not necessary to remove the syringe needle directly under the condition in which the Ringer remains a certain amount, there is no significant problem in the safety of the patient, and the medical team can treat the more urgent patient or service.

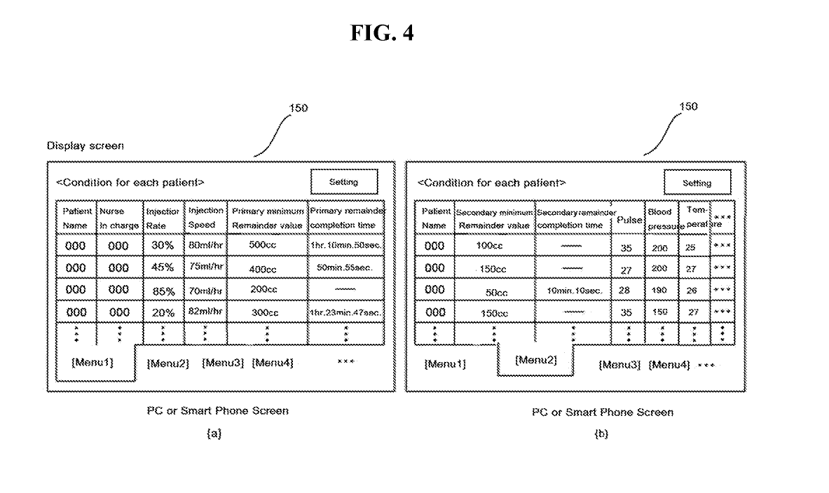

FIG. 4 is a drawing showing in detail a screen of the external instrument 150 by enlarging B portion of FIG. 1. According to what shown in FIG. 4, the living body data of the patient, the remainder amount of the Ringer, the injection speed, the injection completion expectation time or the medical team in charge is displayed on the external instrument 150, and the protector or the medical team can adjust at least one among the remainder amount of the Ringer, the injection speed of the Ringer, the injection completion expectation time of the Ringer through the external instrument 150.

The external instrument 150 is a radio communication instrument and can be realized by a tablet PC or a smart phone or the like. As shown in FIG. 4, the name of the patient, the nurse in charge, the injection rate, the injection speed, the primary and secondary limit of the Ringer, the primary and secondary completion time of the Ringer, the pulse, the blood pressure, the temperature or the like can be set or confirmed at the tablet PC or the smart phone screen being the external instrument 150.

Therefore, the system 100 of giving an injection of Ringer according to the present invention does not require one to go to the hospital room to confirm the Ringer remainder data, the living body data, the injection speed, the injection rate or the injection completion time as well as can adjust the injection speed, the injection rate or the injection completion time while the Ringer is injected by using the external instrument 150.

In addition, if the Ringer remainder amount reach the primary limit or the secondary limit, the external instrument 150 receives the warning signal from the control section 130 and knows this in advance, and inputs the set data according thereto and sends the instruction signal to the control section 130 so as to adjust the injection amount of the Ringer, the injection speed or the injection completion time or interrupt the injection of the Ringer by sending the instruction signal to the control section 130.

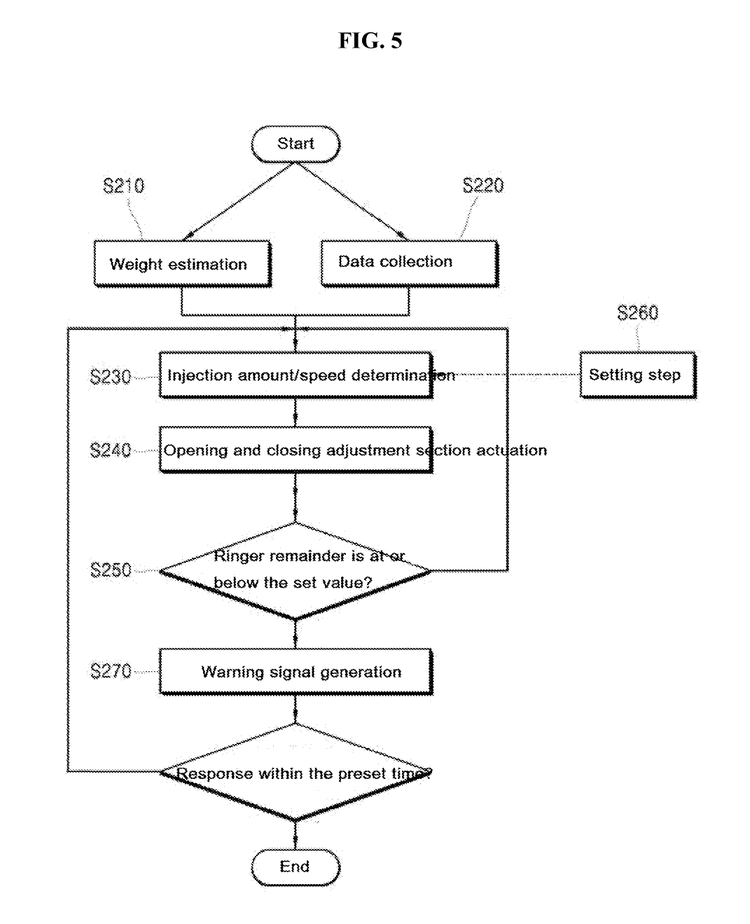

FIG. 5 is a flow diagram showing a method S200 of giving an injection of Ringer according to an embodiment of the present invention. According to what shown in FIG. 5, the method S200 of giving an injection of Ringer using the system 100 of giving an injection of Ringer according to the present invention can comprise: a weight estimation step S210 for measuring the weight of the Ringer bag or estimating the Ringer remainder by a weight estimation section; a data collection step S220 for collecting the living body data of the patient by being connected to the body of the patient by wire or wirelessly; a control step S230 for setting the injection amount or the injection speed of the Ringer by using the data received from the weight estimation step S210 or the data collection step S220; an actuation step S240 for adjusting the actuation condition of an opening and closing adjustment section 140 installed at a Ringer tube according to the injection amount or the injection speed of the Ringer estimated by the control step S230; and a setting step S250 in which the injection amount or the injection speed of the Ringer is set by receiving the input data from an external instrument 150.

The control step S230 can control the injection amount or the injection speed of the Ringer to suit the patient based on the data obtained by the weight estimation step S210 or the data collection step S220. In addition, the control step S230 can set a primary limit and a second limit according to the Ringer remainder or the remainder weight.

The control step S230 can comprise a warning step S270 for warning the external instrument in case where the Ringer remainder or the remainder weight is equal to or below the primary limit or the secondary limit, and a priority discriminating step S260 for discriminating the priority among the input data set by the setting step S250 and the Ringer remainder data estimated by the weight estimation step S210.

The priority discriminating step S260 can preferentially process the input data input from the external instrument in case where the input data are input from the external instrument during the preset time after the warning step S270, and can automatically close the tube opening and closing section or reduce the injection speed of the Ringer by the control step S230 in case where the input data are not input from the external instrument during the preset time.

The method S200 of giving an injection of Ringer according to an embodiment of the present invention follows the order described below.

The medical team inserts the syringe needle to the patient so as to connect the Ringer bag and estimates the Ringer remainder amount data in real time (weight estimation step S210). At this time, a living body data means is connected to the patient by wire or wirelessly and collects the living body data of the patient (data collection step S220). The injection amount or the injection speed of the Ringer is set based on the

Ringer remainder data or the living body data and the signal of the injection amount or the injection speed of the set Ringer is sent to the opening and closing adjustment section (control step S230). The injection amount or the injection speed of the Ringer is adjusted by adjusting the opening and closing of the Ringer tube (actuation step S240). As the Ringer is injected, if the Ringer remainder or the remainder weight falls to or below the preset primary or secondary limit, the warning signal is sent to the external instrument (warning step S270). The input data is set which lets the injection amount or the injection speed of the Ringer to be changed if the medical team or the protector receives the warning signal through the external instrument, or through the information usually received in real time (setting step S250). The injection amount or the injection speed of the Ringer is adjusted by discriminating the priority among the input data and the Ringer remainder data (priority discrimination step S260).

The present invention has been described with reference to limited embodiments and the drawings thereof, however, the present invention is not limited to the embodiments described above, and it will be appreciated by those skilled in the art that various amendments and changes may be made. Therefore, the thought of the present invention shall be comprehended only by the scope of the claim described below, and all the equivalents or the equivalent deformations fall into the scope of the thought of the present invention.

What is claimed is:

1. A system of giving an injection of Ringer comprising:
a weight estimation section for measuring the weight of the Ringer bag or estimating the remainder of the Ringer;
a control section for receiving the data from the weight estimation section and judging and setting an injection amount or an injection speed of the Ringer; and
a Ringer tube opening and closing adjustment section installed at a Ringer tube connected to the Ringer bag and adjusting the injection amount or the injection speed of the Ringer by a signal received from the control section,
wherein the control section comprises a first communication section for controlling an external instrument or an opening and closing adjustment section or for transmitting or receiving a control signal to or from the external instrument or the opening and closing adjustment section, and
wherein the opening and closing adjustment section comprises a second communication section for transmitting or receiving the control signal to or from the external instrument or the control section.

2. The system of claim 1 further comprising a data collection section for collecting living body data of the patient by being connected via wire or wirelessly to the body of the patient and for transmitting the collected living body data to the control section,
wherein the data collection section collects the living body data including the pulse, temperature, electrocardiogram or blood pressure of the patient and transmit it to the control section.

3. The system of claim 2, wherein the control section estimates in real time the injection amount or the injection speed of the Ringer appropriate to the patient in consideration of a Ringer remainder data measured by the weight estimation section or the living body data measured by the data collection section.

4. The system of claim 3, wherein the opening and closing adjustment section comprises a motor actuated according to the Ringer remainder data or the living body data, and a tube opening and closing section which is installed at the Ringer tube so as to adjust the injection amount or the injection speed of the Ringer.

5. The system of claim 4, wherein the external instrument is provided as a radio communication instrument and receives the input data set by the medical team or the protector, and
wherein the first communication section or the second communication section receives the input data and manually adjusts the injection amount or injection speed of the Ringer.

6. The system of claim 5, wherein the control section sets the primary limit or the secondary limit about the Ringer remainder or the remainder weight and sends the warning signal to the external instrument by stages in case where the Ringer is equal to or below the primary limit or the secondary limit.

7. The system of claim 6, wherein the control section comprises the priority setting section which receives the input data from the medical team or the protector during the Ringer injection and discriminates the priority among the input data and the Ringer remainder data measured by the weight estimation section.

8. The system of claim 7, wherein the priority setting section, in case where the input data are input to the external instrument during the preset time after the warning signal is generated, preferentially processes the input data, and in case where the input data are not input to the external instrument during the preset time, automatically closes the tube opening and closing section or reduces the injection speed of the Ringer by the control section.

9. The system of claim 5, wherein the living body data of the patient, the remainder amount of the Ringer, the injection speed, the injection completion expectation time or the medical team in charge is displayed on the external instrument 150, and at least one among the remainder amount of the Ringer, the injection speed of the Ringer, the injection completion expectation time of the Ringer is adjusted by the setting by the protector or the medical team.

10. The system of claim 5 further comprising a data storage section for storing the living body data or the Ringer remainder data transmitted by the first communication section or the second communication section.

11. The system of claim 5, wherein the opening and closing adjustment section further comprises an auxiliary medicine removal/attachment section providing the medicine or injection necessary for the patient, and
wherein the auxiliary medicine removal/attachment section is controlled through the external instrument or by the medical team, protector, or patient.

12. A method of giving an injection of Ringer using the system of giving an injection of Ringer according to claim 8 comprising:
a weight estimation step for measuring the weight of the Ringer bag or estimating the Ringer remainder by a weight estimation section;
a data collection step for collecting the living body data of the patient by being connected to the body of the patient by wire or wirelessly;
a control step for setting the injection amount or the injection speed of the Ringer by using the data received from the weight estimation step and the data collection step;
an actuation step for adjusting the actuation condition of an opening and closing adjustment section installed at a Ringer tube according to the injection amount or the injection speed of the Ringer estimated by the control step; and
a setting step in which the injection amount or the injection speed of the Ringer is set by receiving the input data from an external instrument.

13. The method of claim 12, wherein the control step controls the injection amount or the injection speed of the Ringer to suit the patient based on the data obtained by the weight estimation step or the data collection step.

14. The method of claim 12, wherein the control step sets a primary limit and a second limit according to the Ringer remainder or the remainder weight, and
wherein the control step comprises a warning step for warning the external instrument in case where the Ringer remainder or the remainder weight is equal to or below the primary limit or the secondary limit, and a priority discriminating step for discriminating the priority among the input data set by the setting step and the Ringer remainder data estimated by the weight estimation step.

15. The method of claim 14, wherein the priority discriminating step, in case where the input data are input to the external instrument during the preset time after the warning step, preferentially processes the input data, and in case where the input data are not input to the external instrument during the preset time, automatically closes the tube opening and closing section or reduces the injection speed of the Ringer by the control step.
